(19) United States
(12) Patent Application Publication
(10) Pub. No.: US 2005/0113663 A1
(43) Pub. Date: May 26, 2005

(54) Method and system for automatic extraction of load-bearing regions of the cartilage and measurement of biomarkers

(76) Inventors: Jose Tamez-Pena, Rochester, NY (US); Saara Marjatta Sofia Totterman, Rochester, NY (US)

Correspondence Address: BLANK ROME LLP, 600 NEW HAMPSHIRE AVENUE, N.W., WASHINGTON, DC, 20037, US

(21) Appl. No.: 10/716,934

(22) Filed: Nov. 20, 2003

(51) Int. Cl. G06K 9/00; A61B 5/05

(52) U.S. Cl. 600/407; 600/410; 128/922; 382/128

(57) ABSTRACT

An image is taken of a knee or other region of interest. The cartilage is extracted from the image and is subdivided into load-bearing and non-load-bearing regions. A biomarker is calculated for each of the load-bearing and non-load-bearing regions. The biomarkers can be assessed over time. The biomarkers for the load-bearing and non-load-bearing regions, and their changes, are used to assess the progress of joint disease.

### FIELD OF THE INVENTION

The present invention is directed to a system and method for automatic segmentation of the cartilage of the human knee and more particularly to such automatic segmentation in which the cartilage is subdivided into a plurality of regions, including load-bearing regions and non-load-bearing regions.

### DESCRIPTION OF RELATED ART

The knee joint can be severely affected by osteoarthritis (OA), which is the major cause of disabilities in older people. Furthermore, knee injuries can create immediate major physical impairments via joint instabilities that will affect the joint load distribution or lead to the future development of OA.

In order to minimize the number of people with disabilities, the knee joint has been the focus of several studies that try to understand the knee mechanics and the nature of OA. The knee mechanics studies have focused on understanding the load distributions and the displacements of the knee under static or dynamic loading. Other studies have focused on understanding the joint cartilage and mechanical properties. These mechanical aspects of the joint are three-dimensional (3D); therefore, 3D techniques are preferable over two-dimensional (2D) approaches to analyze the knee mechanical properties.

The paper “Evaluation of Distance Maps from Fast GRE MRI as a Tool to Study the Knee Joint Space” by José G. Tamez-Peña et al, presented at the SPIE Medical Imaging Conference in February, 2003, which is hereby incorporated by reference in its entirety into the present disclosure, documents the state of the art as of that time. The paper teaches a technique for measurement of joint distance. A three-dimensional (3D) method of evaluating the joint space from fast GRE MRI has been developed that allows the reconstruction of the two-dimensional (2D) distance map between the femur and the tibia bone plates. This method uses the MRI data, an automated 3D segmentation, and an unsupervised joint space extraction algorithm that identify the medial and lateral compartments of the knee joint. The extracted medial and lateral compartments of the tibia-femur joint space were analyzed by 2D distance maps, where visual as well quantitative information was extracted. This method was applied to study the dynamic behavior of the knee joint space under axial load. Three healthy volunteers' knees were imaged using fast GRE sequences in a clinical scanner under unloaded (normal) conditions and with an axial load that mimics the person's standing load. Furthermore, one volunteer's knee was imaged at four regular time intervals while the load was applied and at four regular intervals without load. The results show that changes of 50 microns in the average distance between bones can be measured and that normal axial loads reduce the joint space width significantly and can be detected.

A flow chart of the technique disclosed in that paper is shown as FIG. 1. The technique starts in step 102. In step 104, an unsupervised segmentation of fast MRI images is performed. In step 106, the tibia and femur are manually labeled. In step 108, it is determined whether the boundaries of the bone are acceptable. If not, then in step 110, the bone boundaries are corrected using the tracing. Once the bone boundaries are corrected, or of they are determined in step 108 to be acceptable, then in step 112, the bone boundaries are relaxed. In step 114, the weight-bearing volumes are extracted. In step 116, the distance maps are computed. The process ends in step 118.

Thus, measurements of biomarkers such as cartilage volume and cartilage thickness are made over the whole of the cartilage. However, measurements over the whole of the cartilage do not provide complete information concerning the health of the cartilage. For example, the inventors have discovered that in many conditions, the load-bearing regions of the cartilage, which are more stressed, have earlier and more advanced changes in biomarker measurements. The prior art provided no way to detect and assess those earlier and more advanced changes.

The inventors and those working with them have previously proposed techniques for the assessment of various conditions and their change over time by measuring biomarkers. Such techniques are disclosed in WO 03/025837, WO 03/021524, WO 03/012724 and WO 03/009214, whose disclosures are hereby incorporated by reference in their entireties into the present disclosure. However, such techniques do not overcome the above-noted problems of the prior art.

### SUMMARY OF THE INVENTION

It will be apparent from the above that a need exists in the art for a technique for more complete determination of the health of cartilage.

It is therefore an object of the invention to extract subregions from the cartilage.

It is another object of the invention to extract load-bearing and non-load-bearing subregions from the cartilage.

It is still another object of the invention to measure biomarkers of the extracted load-bearing and non-load-bearing subregions.

To achieve the above and other objects, the present invention is directed to a system and method for automatic segmentation of the cartilage of the human knee, from MRI scans, followed by subdivision into a plurality of regions: the load bearing regions which are the medial and lateral load bearing regions; and then the other remaining regions including the trochlear cartilage and the posterior condyle cartilage. Furthermore, the invention then goes on to measure key biomarkers of the load bearing and non-load bearing cartilage, including the cartilage roughness, the cartilage volume (within the different sub-divisions), the cartilage thickness, and the cartilage surface areas. Other biomarkers will be named below.

Segmentation and the measurement of biomarkers, as techniques independent of each other, are known in the art. However, the inventors have discovered that the subdivision of cartilage into load bearing and non-load bearing regions provides a better assessment of the health of the cartilage, since in many conditions the load bearing region, which is more stressed, had earlier and more advanced changes in biomarker measurements. This examination of subregions thereby provides improved diagnostic capability over prior art which would measure biomarkers, such as cartilage volume or thickness, as a whole over the entire cartilage, thus combining information from both load bearing and non-load bearing regions of the cartilage.

### BRIEF DESCRIPTION OF THE DRAWINGS

A preferred embodiment and experimental results therefrom will be set forth in detail with reference to the drawings, in which:

FIG. 1 shows a flow chart of a previous technique for measuring joint spacing;

FIG. 2 shows a flow chart of the technique for cartilage region extraction and biomarker measurement according to the preferred embodiment;

FIG. 3 shows a setup for applying loads to the subject's knee for taking image data;

FIG. 4 shows a schematic diagram of a system for analyzing the image data;

FIGS. 5A-5B show extracted measurements as well as a model of the knee;

FIG. 6 shows results of labeling the weight-bearing volumes;

FIG. 7 shows 3D visualizations of the whole cartilage; and

FIGS. 8A and 8B show visualizations of the cartilage region of interest.

### DETAILED DESCRIPTION OF THE PREFERRED EMBODIMENT

A preferred embodiment of the present invention and experimental results therefrom will be set forth in detail with reference to the drawings, in which like reference numerals refer to like elements throughout.

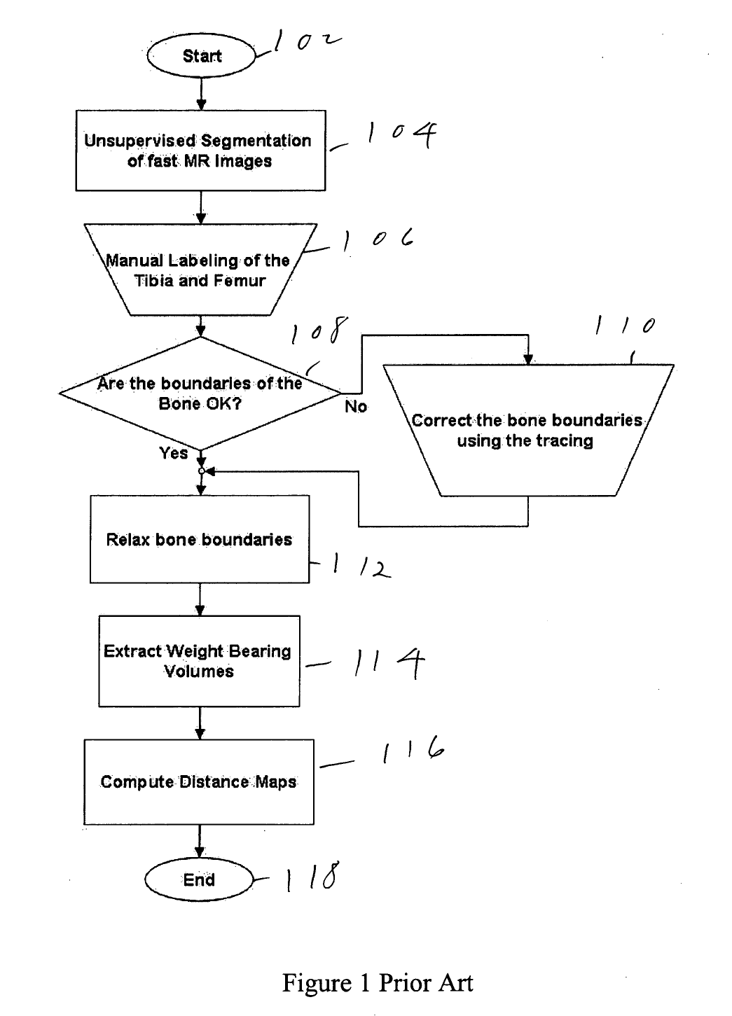

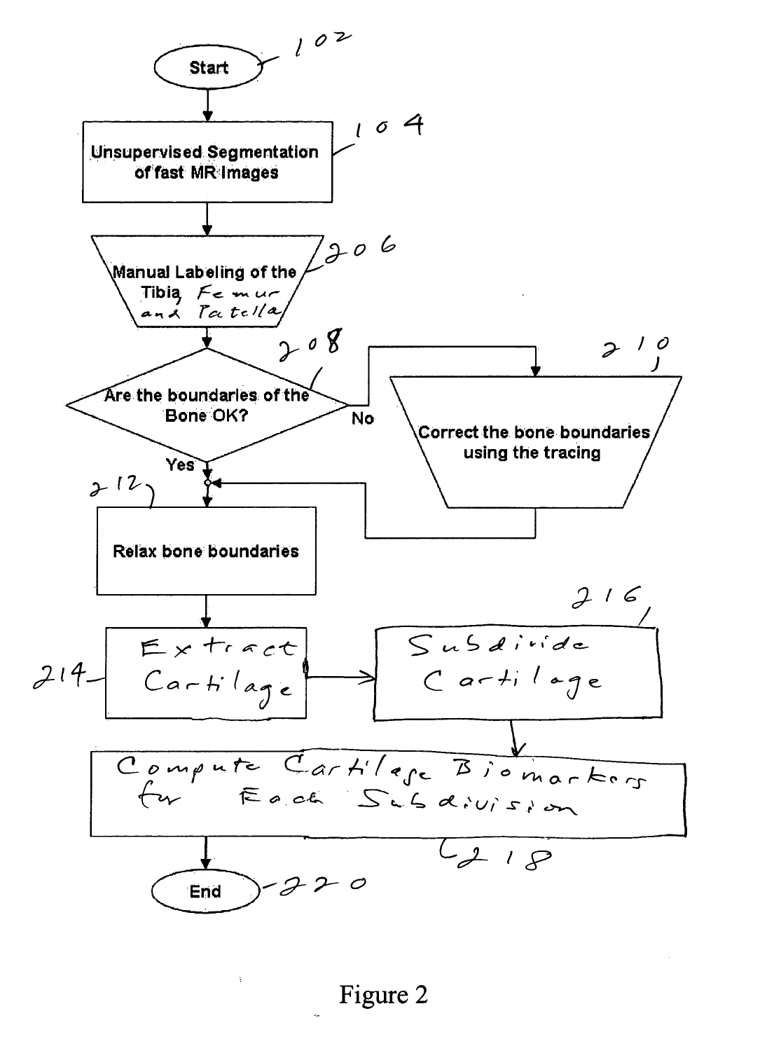

FIG. 2 shows a flow chart of the technique according to the preferred embodiment. Steps 102 and 104 are carried out like steps 102 and 104 of the prior technique of FIG. 1. However, in step 206, the tibia, femur, and patella are manually labeled. Steps 208, 210 and 212 are then carried out essentially like steps 108, 110 and 112 of FIG. 1, except that now the patella is also taken into account.

In step 214, the cartilage is extracted. In step 216, the cartilage is subdivided into subregions, in particular load-bearing and non-load-bearing subregions. In step 218, the cartilage biomarkers are computed for each subregion of the cartilage. The process ends in step 220.

We selected five MR image sets from three healthy adult subjects who had participated in an in vivo magnetic resonance imaging of axial and anterior loads of their knees. The MRI data sets were acquired with the subjects lying in a supine position in a loading device that was designed to comfortably position the knee joint with an average exion angle of 8°, depending on subject height.

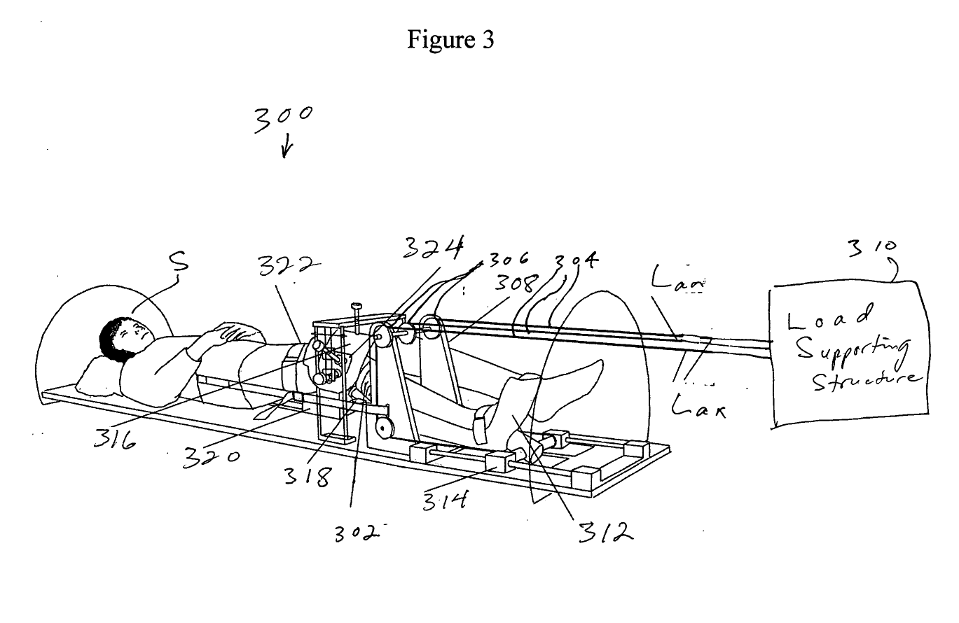

The device 300 is shown in FIG. 3. The device 300 is constructed of non-magnetic, MRI compatible materials. It is designed to rest on top of the existing GE (GE, Milwaukee, Wis.) Signa MRI scanner table and is held in place by the weight of the subject S.

An anterior load Lₐₙ is applied to the proximal tibia by way of a sling 302 fitted around the proximal tibia and attached to a rope 304 and pulleys 306 on a support 308 leading to a structure 310 supporting the applied loads. Axial load Lₐₓ is applied through a foot orthotic 312 attached to a horizontally sliding frame 314. The frame 314 is moved with ropes 304 and pulleys 306 leading to the structure 310 supporting the applied loads. The subject's knee is held in position by a knee wedge 320, a femur strap 322, and condyle cups 324.

A custom-designed four-coil phased array receiver coil including an anterior knee coil 316 and a posterior knee coil 318 was integrated into the loading device 300. The analyzed MRI images were acquired using the same MRI image parameters in a sagittal plane with a 3D fast gradient recalled echo (GRE) sequence (TE: 1.9, TR: 7, 1 Nex, Flip angle: 40°, time of scan 2.05 min.). A 256×256 matrix was used, with a field-of-view of 17 cm and slice thickness of 1.5 mm. Each one of the MRI image sets consisted of a pair of fast GRE MRI scans. The first MRI scan was done on an unloaded knee and was used as a reference. The second MRI scan was done with the subject undergoing an axial load of at least 225 N.

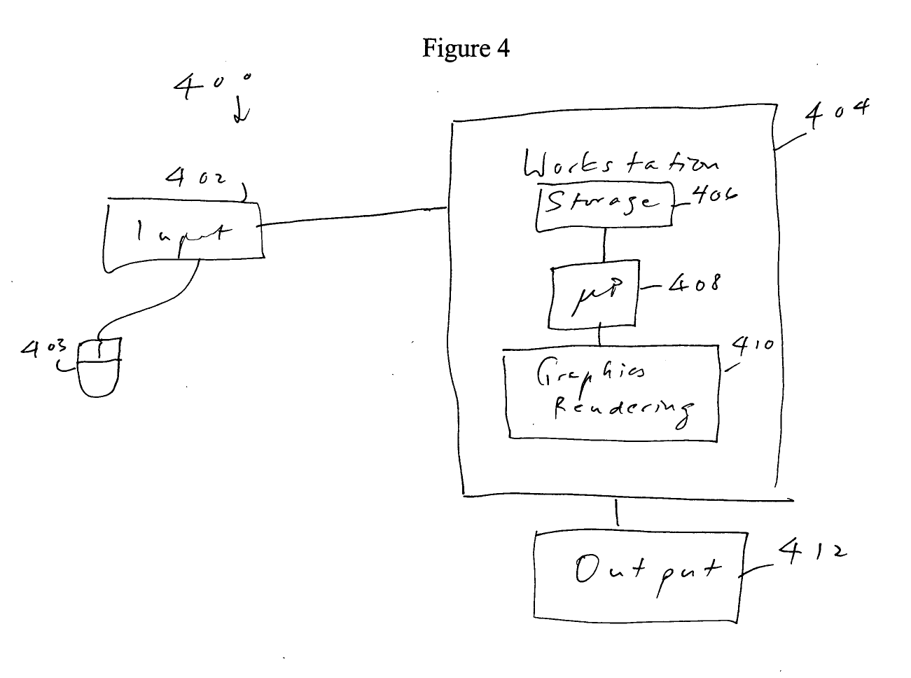

Data analysis was performed with a device such as that of FIG. 4. Device 400 includes an input device 402 for input of the image data, manual tracing input from the user, and the like. The input device can include a mouse 403 or any other suitable tracing device, e.g., a light pen. The information input through the input device 402 is received in the workstation 404, which has a storage device 406 such as a hard drive, a processing unit 408 for performing the processing disclosed above, and a graphics rendering engine 410 for preparing the data for viewing, e.g., by surface rendering. An output device 412 can include a monitor for viewing the images rendered by the rendering engine 410, a further storage device such as a video recorder for recording the images, or both.

Once the image sets were acquired, each one of them was analyzed using an automated method. The first step in the analysis consisted in the accurate extraction of the femur, tibia and patella subchondral bone plates from the Fast GRE MRI data sets. To achieve the desired accuracy we used a three stage supervised approach for the MRI segmentation. First, we use an unsupervised segmentation algorithm (FIG. 2, step 104) which has been used successfully to segment bone structures from standard GRE sequences. Because we were doing the segmentations of fast GRE sequences, the algorithm does not always make accurate estimations of the subchondral bone plates boundaries. Therefore, the second stage consisted of reviewing the segmentation, detecting the errors and correcting those using a tracing tool (FIG. 2, step 206). Once the user has decided that the segmentation of the femur and the tibia appear to be acceptable (FIG. 2, steps 208, 210), we arrive at the third stage: boundary relaxation (FIG. 2, step 212). The boundary relaxation uses a stochastic relaxation technique that uses the information from the segmentation and the MRI data sets to correct the boundary of the segmented structures.

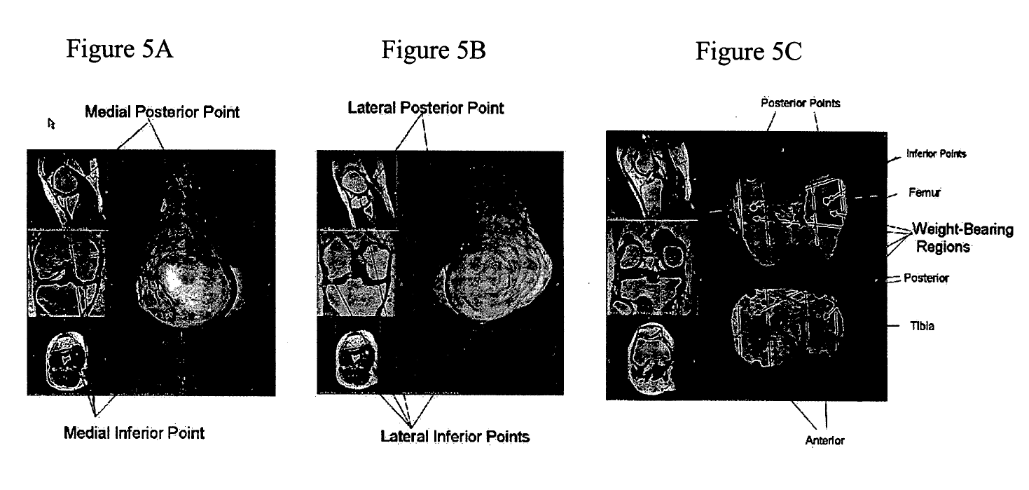

The next step in the analysis of the data consisted of the extraction of the weight bearing volumes (FIG. 2, step 214). For that purpose, we built a very simple parametric model of the knee joint space. This model is based on the unique knee anatomy. The model is seen in FIG. 5C. This model needs the estimation of the knee orientation and the following parameters:
1. width and length of the lateral joint space condyle
2. width and length of the medial joint space condyle

This knee orientation and the eight points are extracted from the segmented tibia and femur using the following approach. First, the most inferior points of the medial and lateral condyle are found by doing a full search on the segmented femur. At the same time the most posterior points of the medial and lateral femur condyles are found. Second, the most posterior points are used to estimate the knee axial rotation. Third, most inferior points are used to estimate the coronal rotation of the femur. Once the axial orientation has been found we proceed to estimate the width of the condyles. Both condyle widths are estimated in the same way: The femur segmentation is searched from the most posterior points toward the anterior position of the inferior points, following the path defined by the orientation. During the search, the width of the condyle is estimated at regular intervals in the orthogonal direction of the axial orientation. Ninety percent of the average measured width is used as the width of the condyle.

Once we have defined the location of the inferior points and the posterior points, we proceed to analyze the tibia segmentation. The tibia segmentation will give us extra information to extract the length of the joint space. For that purpose, we search the tibia in the anterior-posterior direction at the center of the condyle. The extreme anterior points of these searches will define the most anterior location of the joint space. The posterior point of the joint space was defined as sixty-five percent of the distance between the interior point to the posterior point of the condyle.

FIGS. 5A-5C show the extracted measurements. FIG. 5A shows visualization of the posterior and inferior points of medial femur condyle. FIG. 5B shows visualization of the posterior and inferior points of the femur lateral condyle. FIG. 5C shows line segments that define the medial-lateral boundaries of the weight bearing volume.

Once we have found the location, orientation, width and the length of the medial and lateral joint space we proceed to label the joint space (FIG. 2, step 216). The next step in the weight-bearing extraction is the labeling of the weight-bearing regions. This labeling is done using a simple approach. The first step is to identify candidate voxels. The candidate voxels are defined as the voxels that belong to both dilated versions of the tibia and the femur that are not part of the original bone voxels. The dilated versions of the femur and tibia are computed by dilating the surface of the object by a given number. In our experiments we dilated both bones by 9.5 mm. The candidate voxels then are searched and those voxels that are inside the hexahedron defined by the location, orientation, width and length of the medial and lateral joint space are defined as the weight-bearing volumes.

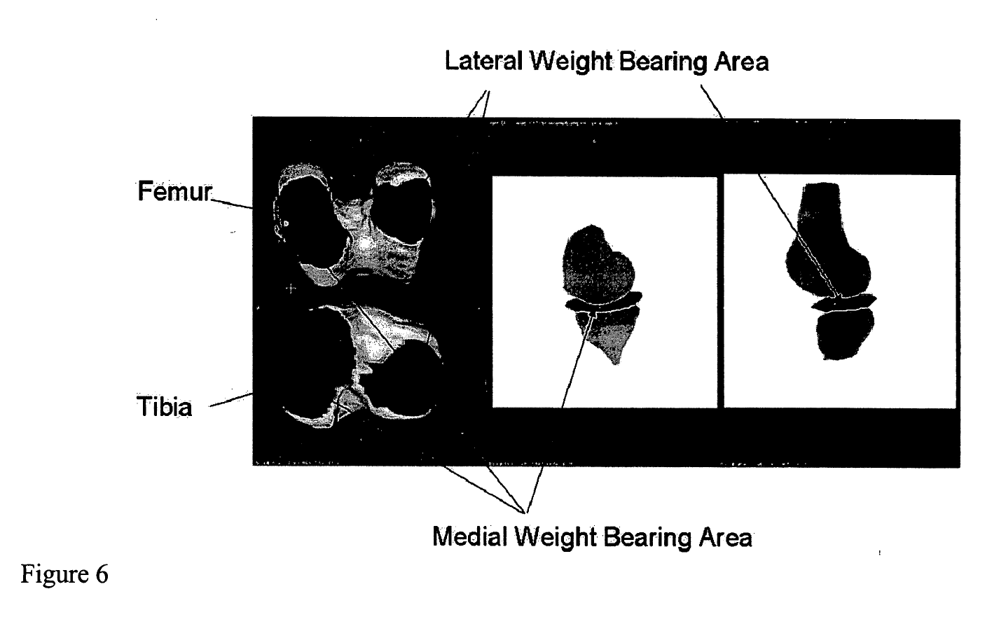

FIG. 6 shows the result of labeling the weight-bearing volumes using our approach. The left part shows the mapping of the weight-bearing contact areas on the femur and the tibia. The middle and right portions show slices through the medial and lateral weight-bearing volumes.

Once the weight-bearing and non-weight-bearing subdivisions of the cartilage are extracted, a cartilage biomarker is computed for each of the subdivisions (FIG. 2, step 218). Biomarkers for use in quantitative assessment of joint diseases and the change in time of joint diseases are taught in the above-cited WO 03/012724, as are methods for extracting and quantifying them.

The computation of biomarkers allows the identification of important structures or substructures, their normalities and abnormalities, and the identification of their specific topological, morphological, radiological, and pharmacokinetic characteristics which are sensitive indicators of joint disease and the state of pathology. The abnormality and normality of structures, along with their topological and morphological characteristics and radiological and pharmacokinetic parameters, are used as the biomarkers, and specific measurements of the biomarkers serve as the quantitative assessment of joint disease.

The following biomarkers are sensitive indicators of osteoarthritis joint disease in humans and in animals and are to be calculated for each subdivision within the cartilage:
cartilage roughness
cartilage volume
cartilage thickness
cartilage surface area
shape of the subchondral bone plate
layers of the cartilage and their relative size
signal intensity distribution within the cartilage layers
contact area between the articulating cartilage surfaces
surface topology of the cartilage shape
intensity of bone marrow edema
separation distances between bones
meniscus shape
meniscus surface area
meniscus contact area with cartilage
cartilage structural characteristics
cartilage surface characteristics
meniscus structural characteristics
meniscus surface characteristics
pannus structural characteristics
joint fluid characteristics
osteophyte characteristics
bone characteristics
lytic lesion characteristics
prosthesis contact characteristics
prosthesis wear
joint spacing characteristics
tibia medial cartilage volume
Tibia lateral cartilage volume
femur cartilage volume
patella cartilage volume
tibia medial cartilage curvature
tibia lateral cartilage curvature
femur cartilage curvature
patella cartilage curvature
cartilage bending energy
subchondral bone plate curvature
subchondral bone plate bending energy
meniscus volume
osteophyte volume
cartilage T2 lesion volumes
bone marrow edema volume and number
synovial fluid volume
synovial thickening
subchondrial bone cyst volume
kinematic tibial translation
kinematic tibial rotation
kinematic tibial valcus
distance between vertebral bodies
degree of subsidence of cage
degree of lordosis by angle measurement
degree of off-set between vertebral bodies
femoral bone characteristics
patella characteristics.

A preferred technique for extracting the biomarkers is with statistical based reasoning as defined in Parker et al (U.S. Pat. No. 6,169,817), whose disclosure is hereby incorporated by reference in its entirety into the present disclosure. A preferred method for quantifying shape and topology is with the morphological and topological formulas as defined by the following references:

Curvature Analysis: Peet, F. G., Sahota, T. S., “Surface Curvature as a Measure of Image Texture” IEEE Transactions on Pattern Analysis and Machine Intelligence 1985 Vol PAMI-7 G:734-738.

Struik, D. J., Lectures on Classical Differential Geometry, 2nd ed., Dover, 1988.

Shape and Topological Descriptors: Duda, R. O, Hart, P. E., Pattern Classification and Scene Analysis, Wiley & Sons, 1973.

Jain, A. K, Fundamentals of Digital Image Processing, Prentice Hall, 1989.

Spherical Harmonics: Matheny, A., Goldgof, D., “The Use of Three and Four Dimensional Surface Harmonics for Nonrigid Shape Recovery and Representation,” IEEE Transactions on Pattern Analysis and Machine Intelligence 1995, 17: 967-981.

Chen, C. W, Huang, T. S., Arrot, M., “Modeling, Analysis, and Visualization of Left Ventricle Shape and Motion by Hierarchical Decomposition,” IEEE Transactions on Pattern Analysis and Machine Intelligence 1994, 342-356.

A higher-order quantitative measure, which can be one or more of curvature, topology and shape, can be made of each joint biomarker.

Of course, the technique described above may be repeated over time so that both the biomarkers and their change over time may be evaluated for the load-bearing and non-load-bearing regions.

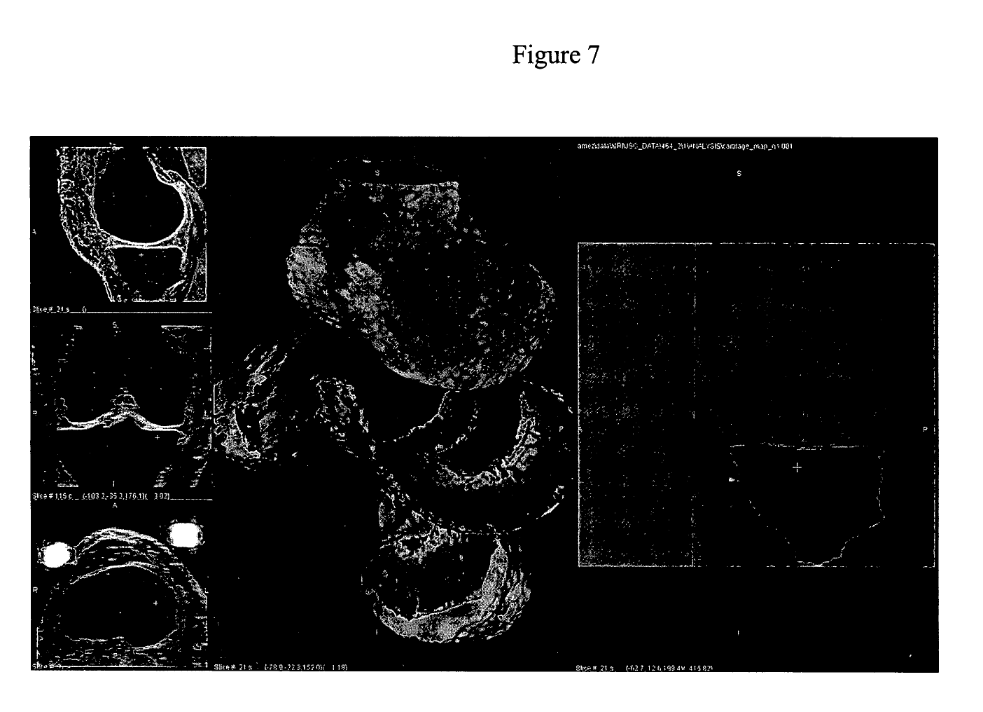

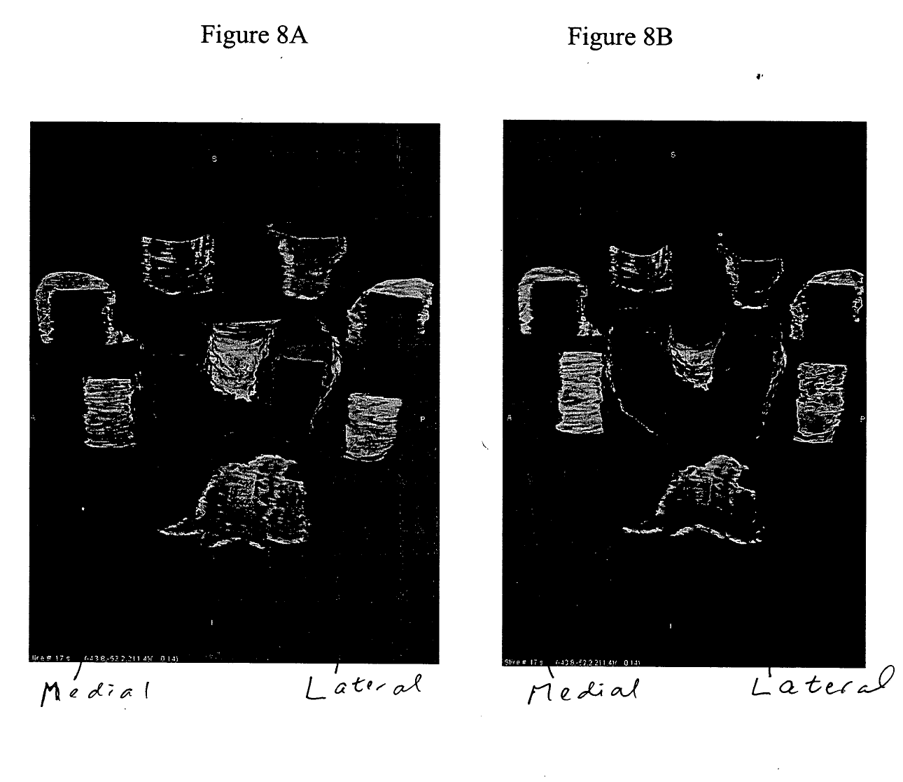

Further results will now be shown in the drawings. FIG. 7 shows 3D visualization of the whole cartilage. FIGS. 8A and 8B show 3D visualization of the cartilage region of interest.

While a preferred embodiment of the present invention has been disclosed, those skilled in the art who have reviewed the present disclosure will readily appreciate that other embodiments can be realized within the scope of the invention. For example, numerical values are illustrative rather than limiting. Also, imaging technologies other than MRI can be used, as can setups for applying load other than that of FIG. 3. Therefore, the present invention should be construed as limited only by the appended claims.

1. A method for evaluating a condition of a region of interest in a patient, the method comprising:
(a) taking image data of the region of interest;
(b) extracting a structure from the image data;
(c) subdividing the structure into load-bearing and non-load bearing subdivisions; and
(d) calculating a biomarker for each of the load-bearing and non-load-bearing subdivisions.

2. The method of claim 1, wherein the region of interest includes a joint.

3. The method of claim 2, wherein the joint is a knee.

4. The method of claim 2, wherein the structure is cartilage in the joint.

5. The method of claim 2, wherein step (a) comprises taking MRI image data.

6. The method of claim 2, wherein step (b) comprises unsupervised segmentation of the image data to provide segmented image data.

7. The method of claim 6, wherein step (b) further comprises manual labeling of bone features in the segmented image data.

8. The method of claim 7, wherein step (b) further comprises determining whether the segmented image data are accurate and, if the segmented image data are not accurate, correcting the segmented image data in accordance with the manual labeling.

9. The method of claim 8, wherein step (b) further comprises relaxing boundaries of the bone features.

10. The method of claim 1, wherein, in step (d), the biomarker comprises a biomarker selected from the group consisting of:
cartilage roughness;
cartilage volume;
cartilage thickness;
cartilage surface area;
shape of the subchondral bone plate;
layers of the cartilage and their relative size;
signal intensity distribution within the cartilage layers;
contact area between the articulating cartilage surfaces;
surface topology of the cartilage shape;
intensity of bone marrow edema;
separation distances between bones;
meniscus shape;
meniscus surface area;
meniscus contact area with cartilage;
cartilage structural characteristics;
cartilage surface characteristics;
meniscus structural characteristics;
meniscus surface characteristics;
pannus structural characteristics;
joint fluid characteristics;
osteophyte characteristics;
bone characteristics;
lytic lesion characteristics;
prosthesis contact characteristics;
prosthesis wear;
joint spacing characteristics;
tibia medial cartilage volume;
tibia lateral cartilage volume;
femur cartilage volume;
patella cartilage volume;
tibia medial cartilage curvature;
tibia lateral cartilage curvature;
femur cartilage curvature;
patella cartilage curvature;
cartilage bending energy;
subchondral bone plate curvature;
subchondral bone plate bending energy;
meniscus volume;
osteophyte volume;
cartilage T2 lesion volumes;
bone marrow edema volume and number;
synovial fluid volume;
synovial thickening;
subchondrial bone cyst volume;
kinematic tibial translation;
kinematic tibial rotation;
kinematic tibial valcus;
distance between vertebral bodies;
degree of subsidence of cage;
degree of lordosis by angle measurement;
degree of off-set between vertebral bodies;
femoral bone characteristics; and
patella characteristics.

11. The method of claim 10, wherein the biomarker further comprises a higher-order mreasure.

12. The method of claim 11, wherein the higher-order measure is selected from the group consisting of curvature, topology and shape.

13. The method of claim 1, wherein steps (a)-(d) are performed at a plurality of times, and wherein the method further comprises (e) determining a change in time in each of the biomarkers calculated in step (d).

14. A system for evaluating a condition of a region of interest in a patient, the system comprising:
an input for receiving an input of image data of the region of interest; and
a processor, in communication with the input, for:
(a) receiving the image data of the region of interest from the input;
(b) extracting a structure from the image data;
(c) subdividing the structure into load-bearing and non-load bearing subdivisions; and
(d) calculating a biomarker for each of the load-bearing and non-load-bearing subdivisions.

15. The system of claim 14, wherein the processor performs step (b) through unsupervised segmentation of the image data to provide segmented image data.

16. The system of claim 15, wherein the input comprises an input for receiving a manual labeling of b one features in the segmented image data, and wherein the processor performs step (b) in accordance with the manual labeling.

17. The system of claim 16, wherein the processor performs step (b) further by whether the segmented image data are accurate and, if the segmented image data are not accurate, correcting the segmented image data in accordance with the manual labeling.

18. The system of claim 17, wherein the processor performs step (b) further by relaxing boundaries of the bone features.

19. The system of claim 14, wherein the biomarker comprises a biomarker selected from the group consisting of:
cartilage roughness;
cartilage volume;
cartilage thickness;
cartilage surface area;
shape of the subchondral bone plate;
layers of the cartilage and their relative size;
signal intensity distribution within the cartilage layers;
contact area between the articulating cartilage surfaces;
surface topology of the cartilage shape;
intensity of bone marrow edema;
separation distances between bones;
meniscus shape;
meniscus surface area;
meniscus contact area with cartilage;
cartilage structural characteristics;
cartilage surface characteristics;
meniscus structural characteristics;
meniscus surface characteristics;
pannus structural characteristics;
joint fluid characteristics;
osteophyte characteristics;
bone characteristics;
lytic lesion characteristics;
prosthesis contact characteristics;
prosthesis wear;
joint spacing characteristics;
tibia medial cartilage volume;
tibia lateral cartilage volume;
femur cartilage volume;
patella cartilage volume;
tibia medial cartilage curvature;
tibia lateral cartilage curvature;
femur cartilage curvature;
patella cartilage curvature;
cartilage bending energy;
subchondral bone plate curvature;
subchondral bone plate bending energy;
meniscus volume;
osteophyte volume;
cartilage T2 lesion volumes;
bone marrow edema volume and number;
synovial fluid volume;
synovial thickening;
subchondrial bone cyst volume;
kinematic tibial translation;
kinematic tibial rotation;
kinematic tibial valcus;
distance between vertebral bodies;
degree of subsidence of cage;
degree of lordosis by angle measurement;
degree of off-set between vertebral bodies;
femoral bone characteristics; and
patella characteristics.

20. The system of claim 19, wherein the biomarker further comprises a higher-order mreasure.

21. The system of claim 20, wherein the higher-order measure is selected from the group consisting of curvature, topology and shape.

22. The system of claim 14, wherein the processor performs steps (a)-(d) at a plurality of times and further performs (e) determining a change in time in each of the biomarkers calculated in step (d).
